(19) United States
(12) Patent Application Publication
(10) Pub. No.: US 2010/0254962 A1
(43) Pub. Date: Oct. 7, 2010

(54) FUNCTIONAL DRINK

(76) Inventor: Karl Zehethofer, Linz (AT)

Correspondence Address: BROOKS KUSHMAN P.C., 1000 TOWN CENTER, TWENTY-SECOND FLOOR, SOUTHFIELD, MI, 48075, US

(21) Appl. No.: 12/739,783

(22) PCT Filed: Oct. 15, 2008
(86) PCT No.: PCT/IB2008/002733

§ 371 Date: Apr. 26, 2010

(30) Foreign Application Priority Data
Oct. 24, 2007 (AT) A 1733/2007

(51) Int. Cl. A61K 31/122; A61P 43/00

(52) U.S. Cl. 424/94.1

(57) ABSTRACT

The invention relates to a liquid food product as aqueous solution with anti-ageing effect, which comprises resveratrole, citric acid, fructose, black grape polyphenol extract, green tea extract, vitamin E, vitamin C and niacin. A concentrated juice from red, edible soft fruit is present at least in a sufficient amount to impart the taste, and advantageously the colour, of the formulation.

The invention relates to a liquid food as an aqueous solution which contains, among other substances, resveratrol, and has what is termed an anti-aging effect.

Since about 2003, the advantageous health effects of resveratrol, its trans isomers (hereinafter for simplicity together called “resveratrol”) and also the substances occurring together with resveratrol in the extract of red grapes have been the subject of great interest.

Resveratrol (“5-[(E)-2-(4-hydroxyphenyl)ethenyl]-benzene-1, 3-diol”, empirical chemical formula: C₁₄H₁₂O₃) is a polyphenol which belongs to the class of stilbenes and is a member of the phytoalexin group. It is a particularly highly active free-radical trap and is therefore able to minimize oxidative stress. It protects from vascular calcification, retards the growth of cancer cells or kills them, reduces the risk of heart infarct, counteracts the aging process and prolongs life, is anti-inflammatory, reduces the cholesterol level, prevents prostate cancer, reduces the risk of stroke, reduces the risk of suffering from colorectal cancer, decreases the risk of suffering from Alzheimer's disease and has antimicrobial and antiviral properties.

EP1648437 A2 (WO 20051002672 A2) proposes, by means of the action of resveratrol in combination with substances such as, for example, niacin, on eukaryotic cells, promoting the expression of the sirtuin genes such as SIRT1, and thereby achieving a life-prolonging anti-aging action. This can proceed not only in vitro, but also on cells in organisms such as animals or humans. The applications for humans are proposed in this case only in the medical sector, for example as an adjuvant in chemotherapy for cancer treatment.

U.S. Pat. No. 6,368,617 B1 proposes a dietary additive for avoiding oxidative stress for the organism of adult humans. The dietary additive contains, in addition to resveratrol, inter alia, also fructose and citric acid, and preferably a mixture of crushed herbs. Preferably, the dietary additive is offered as a powder, typically as a fine green powder, which is stirred into water for consumption and is drunk with this.

DE 102 23 262 A1 describes a coffee substitute. It is said to have the vitalizing action of coffee, but not harmful side effects such as, for example, producing addictive dependency. The water-soluble composition contains, in addition to ginkgo components, in individual proposed examples, green tea extract, grape seed extract and fructose.

In WO 02/081651 A2, a drink is proposed which is said to have the advantageous properties of red wine on the cardiovascular system, essentially owing to the polyphenols present therein, without, therefore, containing alcohol. In addition to resveratrol, the drink also contains catechin from the extract of green tea.

WO 2004/105517 A1 describes an anti-inflammatory drink which, in addition to resveratrol, also contains citric acid, vitamin E, vitamin C and beta-carotene.

U.S. 2006/0251608 A1 describes a combined formulation which consists of two different tablets to be taken 12 hours apart, and also of a cream to be applied topically. The combined formulation serves for curing aged, wrinkled skin. The first tablet contains, inter alia, the substances L-carnitine, red wine grape poly-phenol extract, concentrate of red berry fruits, green tea extract, vitamin C, vitamin E and beta-carotene. The second tablet contains, inter alia, the substances resveratrol, ubiquinone (coenzyme Q10) and selenium.

WO 2007/049796 A2 proposes an anti-aging composition in which resveratrol and red wine grape leaf extract are used. Formulae are proposed for a jelly and for drinks in which, in addition, citric acid and fructose are also present. The anti-aging effect is based in a very isolated manner on resveratrol and is therefore relatively narrow. For use as a drink which can be kept, handled and enjoyed in an uncomplicated manner, additional additives are required.

U.S. 2007/0122502 A1 proposes an anti-aging juice for women, primarily from an age of 35 years, which juice, in addition to the juice of the noni-fruit and components of the ranunculaceae “black cohosh” (Cimicifuga racemosa; it has estrogen-like properties as a medicament), and in addition to a chromium compound and a calcium compound, also contains a plant source of trans-resveratrol. In order to obtain a palatable juice, despite the additions having taste problems, further additives such as fruit juices and citric acid are added.

WO 2007/112366 A2 proposes feeding resveratrol, which can be obtained from various plants, to animals owing to its broad health-promoting action. Administration forms usable by humans are not covered.

In the knowledge of this prior art, the inventor has established the object of providing a formula for a liquid drinkable food as aqueous solution, based on resveratrol, which should permit as well as possible the development of the health-promoting effects, in particular the anti-aging effect, of resveratrol, and in addition contains other substances having “anti-aging” type effects.

In addition, it is important that the food is easy to handle. That is to say:
the active compounds present must be, as a mixture, sufficiently water-soluble, so that no flocculation and/or adhesion or deposits to the package occur,
the food must, as a finished drink without further preservatives and stabilizers, have good keeping quality and be readily transportable in the unrefrigerated state also.

The essential properties which are generally advantageous for use as foods such as appearance (color, viscosity, . . . ), odor, taste, storage life, stability against separation etc., should be achieved to the greatest possible extent not by food additives which are specially targeted for this purpose, but by means of components as close as possible to nature which are required anyway for the anti-aging effect.

In agreement with individual features of the publications mentioned at the outset, for achieving the object, the starting point is a formula in which, in addition to resveratrol, advantageously, citric acid, fructose, red wine grape polyphenol extract, green tea extract, vitamin E, vitamin C and niacin are also present. A juice concentrate of red edible berry fruits is added at least in an amount such that it imparts the flavor to the formula. Preferably, the juice concentrate of red edible berry fruits should also impart the color to the formula.

Red berry fruits which come into consideration are, for example, strawberries, raspberries, blackberries, cranberries, bilberries, redcurrants, cherries, sour cherries, elderberries, red wine grapes.

The red berry fruits are advantageous on the one hand for reasons of presentation, since they facilitate the association linked to red wine grapes which are the essential source of resveratrol and form similar advantageous active substances. In addition, these berry fruits are very popular predominantly for their flavor.

Of great essential importance in the red berry fruits is, however, the fact that these berry types actually do have components that (are known to) have a beneficial effect on the vascular system.

Red berry fruits such as elderberries and bilberries have already been used for centuries in Europe and North America in natural healing. Current studies of the Indiana University School of Medicine have now confirmed the action of their components, flavonoids, polyphenols and anthocyans. A beneficial effect on human health is assigned particularly to the anthocyans which are responsible for the red and blue coloration of the fruits.

A mixture of juice concentrates of elderberries and blackcurrants has been found to be particularly advantageous. These also contain, in addition to the advantageous color, polyphenols, anthocyans and flavonoids. Anthocyans, such as the colorant of elderberries, improve the visual processes, are anti-inflammatory and have a vascular-protective action. In addition, they have a high antioxidant effect and significantly reduce the risk of stroke.

Elderberry retards the aging process of cells by protection of the cell membranes by free radicals and acts as an antioxidant. Elderberry is rich in vitamin C, B, fruit acids, essential oils (have slight sudorific and expectorant actions), flavonoids and the coloring anthocyans. Elderberry assists against colds, kidney and bladder disorders and stomach complaints. In addition, black elderberry contains a violet dye, sambucyanin, which is a secondary plant material and, as an antioxidant, reduces the risk of cancer and cardiovascular disorders.

Redcurrants, in addition to water, contain proteins, carbohydrates, fatty acids and also minerals and anthocyans, flavonoids and catechins (as in green tea). In addition, vitamin C, citric acid and β-caryophyllene occur. β-Caryophyllene is a CB₂ cannabinoid and has actions against inflammation, pain, atherosclerosis and osteoporosis.

The use of red berry fruits is therefore advantageous not only for visual-associative reasons, and for reasons of flavor, but also for scientifically justifiable health reasons.

In addition to the said substances, further components in the food which are advantageous are:

Beta-carotene: It is a precursor of vitamin A and is converted in the body, only as required, into vitamin A, which means that even at a relatively high dose it is not harmful. Vitamin A is of importance for the visual process, for the buildup of dermal structures, promotes the formation of blood corpuscles and plays an important role in metabolism during protein synthesis. Furthermore, carotene is a permitted red-coloring food colorant.

L-Carnitine: This is a naturally occurring vitamin-like substance. It plays an important role in the energy metabolism of human, animal and plant cells. In combination with sport, L-carnitine can act as a fat burning medium.

Ubiquinone coenzyme Q₁₀: This is structurally related to vitamin K and vitamin E. It is used in the mitochondria as an electron and proton carrier in the cellular respiration chain and acts as an antioxidant. An ubiquinone deficit occurs owing to age-related decrease in ubiquinone concentration, physical exertion, stress, elevated alcohol and nicotine consumption and also diseases. Some cholesterol-lowering medicaments (called statins) inhibit the body's own production of ubiquinone. This deficiency can be avoided by ubiquinone taken as a food supplement.

Selenium: As a free-radical trap, this can play an important role in the protection of cell membranes against oxidative destruction.

Niacin (vitamin B3) develops its action much better in combination with vitamin C and E and increases the gene-expression-promoting action of resveratrol on the sirtuin genes.

Also, together with other effects, it causes a considerable improvement of the “anti-aging effect”:

It has a cholesterol—and triglycerol-reducing effect, reduces fat deposits in the tissues, improves the function of the red blood corpuscles and the oxygen transport capacity thereof, which gives rise to an improved brain function. Niacin reduces the cardiovascular risk, and acts as an atheroprotective agent. Niacin is involved in 200 metabolic reactions. Generally, reproduction, protection and repair of cells, of the damage caused by oxidation, total cholesterol is decreased, anti-oxidant activity is developed, particularly in the liver, an important role is played in energy production, blood sugar control and detoxification of environmental poisons via the cytochrome P450 system. Niacin intervenes in a multiplicity of anabolic and catabolic functions in amino metabolism, carbohydrate-fat metabolism, steroid metabolism, vitamin and energy metabolism.

The following advantageous formulation may be mentioned by way of example:

| | | |
|---|---|---|
| Fructose | 110 | g |
| Blackcurrant juice concentrate | 20 | g |
| Elderberry juice concentrate | 14 | g |
| Citric acid | 7 | g |
| L-Carnitine | 1 | g |
| Red wine grape polyphenol extract | 200 | mg |
| Green tea extract | 200 | mg |
| Vitamin C | 120 | mg |
| Resveratrol | 100 | mg |
| Niacin | 36 | mg |
| Vitamin E | 36 | mg |
| Ubiquinone coenzyme Q₁₀ | 10 | mg |
| Beta-carotene | 4 | mg |
| Selenium | 1.5 | μg |

The formula is dissolvable without problem in the mass of water still missing from a total mass of 1 kg. It is storable and transportable without problem at room temperature. In view of the many, in part mutually highly differing, components, it is surprising that no interfering separations or precipitations or other disadvantageous interactions occur.

In the form dissolved in water, the formula is a good-tasting, very healthy ready-to-consume drink of appetizing appearance which can be drunk daily without problem by persons of any sex and age. The drink is simple to produce by weighing together and stirring the corresponding components and because of its properties can be distributed and sold unchilled via customary drink-distribution channels. If, apart from the fructose, no additional crystal sugar (sucrose) is added, this drink is also freely suitable for diabetics.

In addition to meeting the statement of the problem underlying the invention, the food has further advantages:
It does not require any separate substances which are required for an advantageous mouth feel. “Mouth feel” relates, for example, to the following technical properties of a food: density, adhesion, viscosity, moisture properties, graininess.
The food is suitable for diabetics, since, apart from fructose, no artificial or natural sweeteners are required.
The food is suitable for persons suffering from lactose intolerance.
The food does not need gelatin.
The food is simple to keep and transport in PET bottles.
The food is an excellent thirst quencher, since it consists of around 85% pure water and has a fruity refreshing flavor.
The various active components are cross-compatible with one another, that is they do not decrease their respective desired action, but they mutually enhance or supplement one another. This concerns not only the properties as a highly drinkable and highly handleable drink, but also the desired breadth of individual actions for the purpose of forming a food which really earns the name “anti-aging food”.

It may be pointed out here that the described food has essentially the same advantageous actions, not only for humans, but also for other mammals such as, for example, horses, dogs and camels. In this sense, in the patent claims hereinafter, the expression “food” must be taken to mean not only compositions which humans consume orally, but also feeds for mammals.

The patent claims hereinafter relate to the substances mentioned regardless of whether plant extracts or chemically synthesized forms thereof are concerned, and also likewise analogues or mimetics thereof.

1. A liquid food as aqueous solution having anti-aging effect, in the dry matter of which, per kg, the following substances are present in the following mass limits:

| Substance | Minimum | Maximum |
|---|---|---|
| Fructose | 200 g | 800 g |
| Citric acid | 0 g | 100 g |
| L-Carnitine | 0 g | 12 g |
| Red wine grape polyphenol | 0.5 g | 3 g |
| extract | | |
| Green tea extract | 0 g | 3 g |
| Vitamin C | 0 g | 2 g |
| Resveratrol | 0.4 g | 2 g |
| Niacin | 0 g | 0.5 g |
| Vitamin E | 0 g | 0.5 g |

characterized in that, apart from said substances, in the dry matter, per kg, the following further substances are present in the following mass limits:

| Substance | Minimum | | Maximum | |
|---|---|---|---|---|
| Concentrate of red berry fruits | 100 | g | 300 | g |
| Ubiquinone coenzyme Q₁₀ | 0 | mg | 100 | mg |
| Beta-carotene | 0 | mg | 50 | mg |
| Selenium | 0 | μg | 30 | μg |

2. The food as claimed in claim 1, characterized in that, in the dry matter, per kg, the following substances are present in the following mass limits:

| Substance | Minimum | | Maximum | |
|---|---|---|---|---|
| Fructose | 680 | g | 760 | g |
| Blackcurrant juice concentrate | 100 | g | 160 | g |
| Elderberry juice concentrate | 70 | g | 115 | g |
| Citric acid | 35 | g | 55 | g |
| L-Carnitine | 5 | g | 8 | g |
| Red wine grape polyphenol | 1.1 | g | 1.5 | g |
| extract | | | | |
| Green tea extract | 1.1 | g | 1.5 | g |
| Vitamin C | 0.6 | g | 0.9 | g |
| Resveratrol | 0.6 | g | 0.7 | g |
| Niacin | 0.2 | g | 0.3 | g |
| Vitamin E | 0.2 | g | 0.3 | g |
| Q 10 | 45 | mg | 85 | mg |
| Beta-carotene | 20 | mg | 30 | mg |
| Selenium | 0 | μg | 15 | μg |
